(19) United States
(12) Patent Application Publication
(10) Pub. No.: US 2026/0306436 A1
(43) Pub. Date: Oct. 8, 2026

(54) TRETINOIN LIPOSOME FORMULATIONS AND USES THEREOF TO TREAT CANCER

(71) Applicant: Highfield BioPharmaceuticals Corporation, Hangzhou, Zhejiang (CN)

(72) Inventors: Yuhong XU, Hangzhou, Zhejiang (CN); Anjie ZHENG, Hangzhou, Zhejiang (CN); Yongchao DAI, Hangzhou, Zhejiang (CN); Jianmin LIU, Shanghai (CN); Shanshan JIN, Hangzhou, Zhejiang (CN); Fengwei XU, Hangzhou, Zhejiang (CN); Xuan WU, Hangzhou, Zhejiang (CN); Xun WANG, Hangzhou, Zhejiang (CN); Wenjun GUO, Hangzhou, Zhejiang (CN); Yongfeng HUANG, Shanghai (CN)

(73) Assignee: Highfield BioPharmaceuticals Corporation, Hangzhou, Zhejiang (CN)

(21) Appl. No.: 19/482,527

(22) PCT Filed: May 7, 2024
(86) PCT No.: PCT/IB2024/054455

§ 371 Date: Nov. 7, 2025

(30) Foreign Application Priority Data
May 9, 2023 (CN) 202310535456.7
Jun. 8, 2023 (CN) 202310679399.X

(51) Int. Cl. A61K 31/203; A61K 9/1271; A61P 35/00

(52) U.S. Cl. CPC A61K 31/203; A61K 9/1271; A61P 35/00

(57) ABSTRACT

Described are methods for treating cancer an effective amount of all-trans retinoic acid (ATRA) disposed in a liposome.

### RELATED APPLICATIONS

This application claims priority from Chinese Application No. 2023105354567 filed May 9, 2023, and Chinese Application No. 202310679399X filed Jun. 8, 2023, each of which are incorporated herein by reference in their entireties.

### BACKGROUND

Retinoic acid is a metabolite of vitamin A in the body. All-trans retinoic acid (ATRA) is used as a drug to treat acne, and is also an important drug for the clinical treatment of acute promyelocytic leukemia (APL). All-trans retinoic acid (ATRA) affects gene expression by binding to specific receptors (RARs, RXRs and RORs) in cells, and promotes APL cell differentiation and PML/RARa gene in the treatment of acute promyelocytic leukemia degradation, to achieve the effect of treatment.

However, the clinical application of all-trans retinoic acid drugs is limited by the following aspects: all-trans retinoic acid has very low water solubility, all-trans retinoic acid plasma half-life is short, and its efficacy requires maintaining a certain concentration of the drug in the target organ. Therefore, it is particularly important to choose a drug delivery method suitable for all-trans retinoic acid.

Liposomes have been used as a nano drug delivery vehicle for more than 30 years. A variety of anticancer drugs based on liposome delivery systems have been widely used in clinical treatment of tumors. The most successful drug is doxorubicin liposome. The advantage of liposome as a drug delivery system is that it changes the biodistribution of the drug, reduces the systemic toxicity of the drug, and can achieve a long circulation/targeting effect, thus increasing the drug concentration of the target tissue.

Most of the previously reported all-trans retinoic acid liposome formulations incorporate all-trans retinoic acid into the phospholipid bilayer. Due to the physical properties of all-trans retinoic acid, the all-trans retinoic acid liposome produced by this method is not ideal in terms of drug loading and stability in vivo.

### SUMMARY

The present disclosure includes methods for treating cancer, comprising administering to a subject in need thereof an effective amount of all-trans retinoic acid (ATRA) formulated (disposed) in a liposome comprising an inner liposome aqueous phase comprising, ATRA and an aqueous solution of calcium acetate having a pH of 7 to about 10, wherein lipid components of the liposome comprise hydrogenated soybean phosphatidylcholine (HSPC), cholesterol (CHOL), and distearoylphosphatidylethanolamine-polyethylene glycol 2000 (DSPE-PEG2000).

In another embodiment, at least a portion of the ATRA present in the inner liposome aqueous phase is crystalline and has phase change and/or a melting point of the ATRA in the liposome that is lower than the melting point of bulk solid ATRA. While not being limited to the theory, this provides evidence of the crystalline structures within the liposome.

Preferably the inner liposome aqueous phase is about pH 8.5 to about pH 9.5, and a particularly preferred embodiment it is about pH 9.

In some embodiments, the cancer is brain cancer. In some embodiments, the brain cancer is glioma. In some embodiments, the glioma is glioblastoma or oligodendroglioma. In some embodiments, the glioma is recurrent or progressive glioma. In some embodiments, the recurrent or progressive glioma is recurrent or progressive glioblastoma.

### BRIEF DESCRIPTION OF THE DRAWINGS

FIG. 1 is a graph that shows the results from a Nano-DSC test of all-trans retinoic acid (ATRA) liposomes with drug-to-lipid ratio=0.1 and different loading times.

FIG. 2 is a graph that shows the results from a Nano-DSC test of all-trans retinoic acid (ATRA) liposomes with drug-to-lipid ratio=0.2 and different loading times.

FIG. 3 is a graph that shows the results from a Nano-DSC test of all-trans retinoic acid (ATRA) liposomes with drug-to-lipid ratio=0.25 and different loading times.

FIG. 4 is a graph that shows the results from a Nano-DSC test of all-trans retinoic acid (ATRA) liposomes with drug-to-lipid ratio=0.3 and different loading times.

FIG. 5 is a graph showing ATRA plasma concentration measurement in patients after receiving a single dose (SD) and multiple doses (MD) of HF1K16.

FIG. 6 is a graph showing the percentage change of MDSCs in PBMCs of patients during treatment, wherein C1D1 indicates cycle 1 day 1 and C6D1 indicates cycle 6 day 1.

### DETAILED DESCRIPTION

The protection scope of the present disclosure is not limited to the specific embodiments described below; the terms used in the embodiments of the present disclosure are intended to describe the specific embodiments, and not to limit the protection scope of the present disclosure. The test methods which do not specify the specific conditions in the following examples are usually carried out according to conventional conditions or according to the conditions recommended by each manufacturer.

All technical and scientific terms used in the present disclosure have the same meaning as commonly understood by those skilled in the art, unless otherwise defined. In addition to the specific methods, devices, and materials used in the embodiments, any method, device, and material in the prior art that are similar to or equal to that described in the embodiments of the present disclosure may also be used to implement the present disclosure according to the prior art mastered by those skilled in the art and the description of the present disclosure.

When the numerical values are given by the examples, it should be understood that the two endpoints of each numerical range and any one of the two endpoints may be selected, unless otherwise described in the present disclosure.

Unless otherwise stated, the experimental methods, detection methods, and preparation methods disclosed in the present disclosure all adopt conventional molecular biology, biochemistry, chromatin structure and analysis, analytical chemistry, cell culture, and recombinant DNA technology in the art and conventional technology in related fields. These techniques are well described in the prior literature, for specific detail, referring to Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999, and the like.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosure.

The present disclosure provides an all-trans retinoic acid (ATRA) liposome formulation comprising ATRA disposed in a liposome. The lipid components of the liposome, which may form a bilayer, comprise a phosphatidylcholine (e.g., hydrogenated soybean phosphatidylcholine (HSPC), distearoylphosphatidylcholine (DSPC), or dipalmitoylphosphatidylcholine (DPPC)), cholesterol (CHOL), and distearoylphosphatidylethanolamine-polyethylene glycol 2000 (DSPE-PEG2000).

In some embodiments, the molar ratio among the phosphatidyl choline, e.g., HSPC, CHOL, and DSPE-PEG2000 is in a range of (30-80):(0.1-40):(0.1-30). In some embodiments, the molar ratio among HSPC, CHOL, and DSPE-PEG2000 is about 57:38:5. In some embodiments, the molar ratio among HSPC, CHOL, and DSPE-PEG2000 is about 55:40:5.

In some embodiments, the molar ratio among DSPC, CHOL, and DSPE-PEG2000 is in a range of (30-80):(0.1-40):(0.1-30). In some embodiments, the molar ratio among DPSC, CHOL, and DSPE-PEG2000 is about 57:38:5. In some embodiments, the molar ratio among DSPC, CHOL, and DSPE-PEG2000 is about 55:40:5.

In some embodiments, the molar ratio among DPPC, CHOL, and DSPE-PEG2000 is in a range of (30-80):(0.1-40):(0.1-30). In some embodiments, the molar ratio among DPPC, CHOL, and DSPE-PEG2000 is about 57:38:5. In some embodiments, the molar ratio among DPPC, CHOL, and DSPE-PEG2000 is about 55:40:5.

The inner liposome aqueous phase comprises ATRA and an aqueous solution of calcium acetate having a pH of 7 to about 10.

All-trans-retinoic acid, also known as ATRA, is represented by (I):

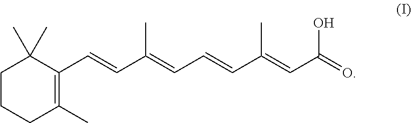 [SMILES: CC1=C(/C=C/C(C)=C/C=C/C(C)=C/C(=O)O)C(C)(C)CCC1]

At least a portion of the ATRA is present in the inner liposome aqueous phase of the liposome in crystalline form, e.g., as an ATRA-calcium pseudopolymorph, the structure of which is shown in (II),

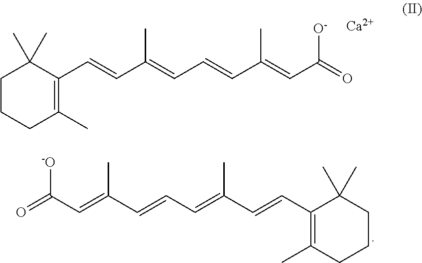

Accordingly, this portion of the ATRA may be suspended in the aqueous solution of calcium acetate.

In some embodiments, the ATRA is present in the inner liposome aqueous phase at a concentration of at least about 2 mg/mL. In some embodiments, the ATRA is present in the inner aqueous liposome at a concentration of about 2 mg/mL to about 30 mg/mL. In some embodiments, the ATRA is present in the inner liposome aqueous phase at a concentration of about 2 mg/mL to about 25 mg/mL. In some embodiments, the ATRA is present in the inner liposome aqueous phase at a concentration of about 2 mg/mL to about 20 mg/mL. In some embodiments, the ATRA is present in the inner liposome aqueous phase at a concentration of about 2 mg/mL to about 15 mg/mL. In some embodiments, the ATRA is present in the inner liposome aqueous phase at a concentration of about 2 mg/mL to about 10 mg/mL. In some embodiments, the ATRA is present in the inner liposome aqueous phase at a concentration of about 2 mg/mL to about 5 mg/mL. In some embodiments, the ATRA is present in the inner liposome aqueous phase at a concentration of about 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, or even higher. These concentrations do not include any ATRA that may be disposed in the liposome other than in the inner liposome aqueous phase.

In some embodiments, the molar ratio of ATRA to the lipid components ranges from about 1 to about 20. In some embodiments, the molar ratio of ATRA to the lipid components ranges from about 1 to about 5.

In some embodiments, the concentration of calcium acetate in the aqueous solution of calcium acetate is 100 mM to 500 mM. In some embodiments, the concentration of calcium acetate in the aqueous solution of calcium acetate is 120 mM to 360 mM.

The pH of the aqueous solution of calcium acetate in the inner liposome aqueous phase is of 7 to about 10. In some embodiments, the pH of the aqueous solution of calcium acetate is about 8 to about 10. In some embodiments, the pH of the aqueous solution of calcium acetate is about 8.5 to about 9.5. In some embodiments, the pH of the aqueous solution of calcium acetate is about 7.5. In some embodiments, the pH of the aqueous solution of calcium acetate is about 8. In some embodiments, the pH of the aqueous solution of calcium acetate is about 8.5. In some embodiments, the pH of the aqueous solution of calcium acetate is about 9. In some embodiments, the pH of the aqueous solution of calcium acetate is about 9.5. In some embodiments, the pH of the aqueous solution of calcium acetate is about 10.

In some embodiments, the average particle size of the liposome is about 30 nm to about 200 nm. In some embodiments, the average particle size of the liposome is about 50 nm to about 150 nm. In some embodiments, the average particle size of the liposome is about 70 nm to about 130 nm. In some embodiments, the average particle size of the liposome is about 50 nm to about 100 nm. In some embodiments, the average particle size of the liposome is about 80 nm.

The ATRA may be loaded into pre-formed liposomes by way of a calcium acetate gradient, which is an active drug loading method. Broadly, the method entails using a composition comprising: a) all-trans retinoic acid (ATRA), optionally further comprising a solubilizing agent, b) a buffer solution, c) a blank liposome (i.e., a liposome that does not contain ATRA) comprising an inner liposome aqueous phase containing an aqueous solution of calcium acetate having a pH of about 7 to about 10.

In some embodiments (and referring to liposome in the plural), the calcium acetate gradient method comprises the following operations or steps: 1) taking all raw materials (lipids) for preparing liposomes in the desired amounts and concentrations, and adding ethanol to obtain an ethanol mixture; 2) adding (e.g., injecting the ethanol mixture produced in operation 1) to an aqueous solution of calcium acetate having a pH of 7 to about 10, thus producing blank liposome vesicles that contain the calcium acetate solution (i.e., the inner phase); 3) extruding the blank liposome vesicles obtained in operation (2) through polycarbonate membranes to obtain a composition containing blank (i.e., ATRA-free) liposomes contain the aqueous solution of calcium acetate; 4) dialyze to remove the calcium acetate from the outer phase and place the blank liposomes into an isotonic liquid (e.g., 7.5% sucrose buffer with 10 mM histidine, 5% vitamin C (w/w) and 0.01% EDTA-NA or 0.9% NaCl (w/w) with 10 mM HEPES) having a pH of about 6.0 to about 8.0, to obtain blank liposomes having a calcium acetate gradient between an inner aqueous phase and an outer aqueous phase, e.g., wherein the pH of the inner phase is greater than the outer phase and ranges from about 7 to about 10; 5) preparing a solution of all-trans retinoic acid (ATRA) in a buffer, which optionally contains a solubilizing agent; 6) adding an all-trans retinoic acid solution to the blank liposomes having the calcium acetate gradient between the inner aqueous phase and the outer aqueous phase obtained in operation (4); 7) incubating the blank liposome/ATRA buffer solution/suspension formed in operation 6), and removing free all-trans retinoic acid not uploaded into the liposomes, to obtain the all-trans retinoic acid liposomes.

In some embodiments, in operation (2), the pH of the aqueous solution of calcium acetate in the inner liposome aqueous phase is about 8.5 to about 9.5. In some embodiments, in operation (2), the pH of the aqueous solution of calcium acetate in the inner liposome aqueous phase is about 9. In some embodiments, in operation (2), the pH of the aqueous solution of calcium acetate in the inner liposome aqueous phase is about 9.5. In some embodiments, in operation (2), the concentration of calcium acetate in the aqueous solution of calcium acetate is 100 mM to 500 mM. In some embodiments, in operation (2), the concentration of calcium acetate in the aqueous solution of calcium acetate is 200 mM to 400 mM.

In some embodiments, the buffer solution in (5) above further comprises a solubilizing agent for increasing solubility of the all-trans retinoic acid. In some embodiments, the solubilizing agent is any one or more of dimethylsulfoxide (DMSO), polyvinylpyrrolidone (PVP), hydroxypropyl methylcellulose (HPMC), cyclodextrin, and polyethylene glycol (PEG). In some embodiments, the cyclodextrin is hydroxypropyl-β-cyclodextrin (HP-β-CD). In general, the weight ratio of the solubilizing agent to ATRA ranges from about 5:1 to about 50:1 (w/w). In some embodiments, the weight ratio of the cyclodextrin (e.g., hydroxypropyl-β-cyclodextrin) to ATRA is from 5:1 to 50:1 (w/w).

In some embodiments, the incubating in operation (7) is conducted for about 15 minutes to about 1 hour. In some embodiments, the incubating is conducted for about 45 minutes. In some embodiments, the incubating is conducted for about 30 minutes.

In some embodiments, the incubation in operation (7) is conducted at a temperature of about 20° C. to about 40° C. In some embodiments, the incubation is conducted at a temperature of about 25° C. to about 37° C. In some embodiments, the incubation is conducted for about one hour or less, at a temperature of about 25° C.

### Pharmaceutical Compositions

The ATRA liposomes may be formulated into a pharmaceutical composition that includes an effective amount of the ATRA liposomes and a pharmaceutically acceptable carrier. The term “pharmaceutically acceptable carrier,” as known in the art, refers to a pharmaceutically acceptable material, composition or vehicle, suitable for administering the ATRA liposomes to mammals. Suitable carriers may include, for example, liquids (both aqueous and non-aqueous alike, and combinations thereof), that function to carry or transport the ATRA liposomes from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, the ATRA liposomes may be formulated into a given type of composition in accordance with conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the parenteral (e.g., subcutaneous (s.c.), intravenous (i.v.), intramuscular (i.m.), and intrasternal injection) administration. Parenteral (e.g., intrathecal, subcutaneous, intravenous, intraventricular, intramuscular, or intraarterial injection, either bolus or infusion which may be continuous or non-continuous) administration may be advantageous in that the ATRA liposomes may be administered relatively quickly such as in the case of a single-dose treatment and/or an acute condition.

In some embodiments, the ATRA liposomes are formulated for intravenous administration (e.g., systemic intravenous injection). In some embodiments, the intravenous fusion time is about 60 to about 90 minutes. In some embodiments, the ATRA liposomes are formulated for intrathecal administration.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in an isotonic saline or glucose solution. Among the acceptable vehicles and solvents that may be employed are water, Ringer's solution, U.S.P. and isotonic sodium chloride solution. In addition, sterile, fixed oils are conventionally employed as a solvent or suspending medium. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

### Methods of Use

In some aspects, the present disclosure is directed to treating cancer. The methods entail administering an effective amount of ATRA, to a subject in need thereof.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from a specific disease or disorder, and subjects suspected of suffering from a specific disease or disorder are not necessarily two distinct groups.

In some embodiments, the cancer is a thymic epithelial tumor, ovarian adenocarcinoma, duodenal carcinoma, bile duct cancer, small cell lung cancer, breast cancer, gastric cancer, or pancreatic cancer.

In some embodiments, the cancer is brain cancer. In some embodiments, the brain cancer is a glioma. In some embodiments, the glioma is astrocytoma, glioblastoma, oligodendroglioma, or ependymoma. In some embodiments, the glioma is recurrent or progressive glioma. In some embodiments, the recurrent or progressive glioma is recurrent or progressive glioblastoma.

ATRA may be administered to a patient, e.g., a cancer patient, as a monotherapy or by way of combination therapy, and as a front-line therapy or a follow-on therapy for patients who are unresponsive to front line therapy. Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments, and the subject was refractory to the treatment(s) or wherein the cancer has reoccurred; or as “third-line”, “fourth-line”, etc. treatments, either alone or in combination with other treatments. Therapy may also be given to patients who have had previous treatments which have been partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, ATRA may be administered to a patient who has received another therapy, such as chemotherapy, radioimmunotherapy, surgical therapy, immunotherapy, radiation therapy, targeted therapy or any combination thereof.

In some embodiments, the treatment methods of the present disclosure entail at least 6-week treatment wherein the administration of ATRA to the patient occurs in a single dose once every other day for 1 or 2 weeks, followed by a 1-week off period, wherein the combination of the 1 or 2-week treatment and the 1-week off period constitutes a treatment cycle. Therefore, each treatment cycle lasts for 2 or 3 weeks. The number of treatment cycles is variable and may continue after complete remission. In some embodiments, the treatment cycles may range from 1 to about 10 cycles. In some embodiments, the method of treatment comprises at least two 3-week treatment cycles. In some embodiments, the method of treatment comprises at least three 2-week treatment cycles.

As used herein, the term, “effective amount” refers to an amount of ATRA that is effective in producing the desired response in a particular patient suffering from cancer. The term “effective amount” thus includes the amount of ATRA, that when administered, induces a positive modification in the cancer to be treated, or is sufficient to prevent or at least inhibit progression of the cancer, or alleviate to some extent, one or more of the symptoms of the cancer being treated in a subject, or which simply kills or inhibits the growth of cancerous cells.

The total daily dosage of ATRA and usage thereof may be decided in accordance with standard medical practice, e.g., by the attending physician using sound medical judgment. The specific an effective dose for any particular subject may depend upon a variety of factors including the disease or disorder being treated and the severity thereof (e.g., its present status); the age, body weight, general health, sex and diet of the subject; the time of administration, route of administration, and rate of excretion of ATRA employed; the duration of the treatment; drugs used in combination or coincidental with ATRA; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, (2001)).

In some embodiments, the dosages is based on body surface area, and may range from about 45 mg per m² to about 160 mg per m² of the subject's body surface area. In some embodiments, the ATRA liposomes are administered at a dose of 45 mg ATRA/m² of the subject's body surface area. In some embodiments, the ATRA liposomes are administered at a dose of 90 mg ATRA/m² of the subject's body surface area. In some embodiments, the ATRA liposomes are administered at a dose of 120 mg ATRA/m² of the subject's body surface area. In some embodiments, ATRA is administered at a dose of 160 mg per m² of the subject's body surface area.

In some embodiments, the dosage is a flat dose (or “dosage”). In some embodiments, the dosage may range from about 60 mg to about 350 mg. In some embodiments, the dosage may range from about 80 mg to about 300 mg. In some embodiments, the dosage may range from about 80 mg to about 240 mg, e.g., about 80, about 90, about 100, about 110, about 120, about 130, about 140, about 150, about 160, about 170, about 180, about 190, about 200, about 210, about 220, about 230, or about 240 mg. In some embodiments, the dosage may range from about 120 mg to about 240 mg. In some embodiments, the dosages may range from about 160 mg to about 200 mg. In some embodiments, the dosage may range from about 65 to about 90 mg. In some embodiments, the dosage may range from about 135 to about 180 mg. In some embodiments, the dosage may range from about 120 mg to about 180 mg, e.g., about 120, about 130, about 140, about 150, about 160, about 170, or about 180 mg. In some embodiments, the dosage may range from about 180 to about 240 mg. In some embodiments, the dosage may range from about 240 to about 320 mg. In some embodiments, the dosage is about 280 mg. In some embodiments, the dosage is about 210 mg. In some embodiments, the dosage is about 180 mg. In some embodiments, the dosage is about 160 mg, e.g., about 157.5 mg. In some embodiments, the dosage is about 120 mg. In some embodiments, the dosage is about 80 mg, e.g., about 78.75 mg.

### Combination Therapy

ATRA liposomes may be used in combination or concurrently with at least one other active ant-cancer agent or therapeutic As used herein, the terms “in combination” and “concurrently are used interchangeably and mean that the agents are co-administered, which includes substantially contemporaneous administration, by way of the same or separate dosage forms, and by the same or different modes of administration, or sequentially, e.g., as part of the same treatment regimen, or by way of successive treatment regimens. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

The dosage of the additional anti-cancer therapeutic may be the same or lower than known or recommended doses. See, Hardman et al., eds., Goodman & Gilman's The Pharmacological Basis Of Therapeutics, 10th ed., McGraw-Hill, New York, 2001; Physician's Desk Reference 60th ed., 2006. Anti-cancer agents that may be used in combination with the bispecific compounds are known in the art. See, e.g., U.S. Pat. No. 9,101,622 (Section 5.2 thereof) and U.S. Pat. No. 9,345,705 B2 (Columns 12-18 thereof). Representative examples of types of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), anti-angiogenic agents, therapeutic antibodies (e.g., mono-specific and bispecific antibodies) and CAR-T therapy.

In some embodiments, the ATRA liposomes are used in combination with one or more of a programmed cell death protein (PD-1) inhibitor, a programmed cell death ligand 1 (PD-L1) inhibitor, a cytotoxic T lymphocyte associated protein 4 (CTLA4) inhibitor, a chimeric antigen receptor (CAR)-immune (e.g., T cell), or a cancer vaccines.

Representative examples of PD-1 inhibitors include pembrolizumab, nivolumab, cemiplimab, dostarlimab, retifanlimab, vopratelimab, spartalizumab, camrelizumab, sintilimab, tislelizumab, toripalimab, AMP-224, AMP-514, and acrixolimab.

Representative examples of PD-L1 inhibitors include atezolizumab, avelumab, durvalumab, KN035, cosibelimab, AUNP12, CA-170, and BMS-986189.

Representative examples of CTLA-4 inhibitors include ipilimumab and tremelimumab.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1: All-Trans Retinoic Acid Liposomes

The lipid components of the present liposome are commercially available. For example, hydrogenated soybean phosphatidylcholine (HSPC) was purchased from NOF Corporation, pegylated phospholipid: distearoylphosphatidylethanolamine-polyethylene glycol 2000 (DSPE-PEG2000), and cholesterol were purchased from Avanti Polar Lipids; All-trans retinoic acid was purchased from Sigma.

Preparation of all-Trans Retinoic Acid Liposomes
(1) Hydrogenated soybean phosphatidylcholine (HSPC), distearoylphosphatidylethanolamine-polyethylene glycol 2000 (DSPE-PEG2000), and cholesterol (CHOL) were mixed at a weight ratio of 3:1:1 in ethanol.
(2) The ethanol mixture obtained in step (1) was added to a calcium acetate buffer solution (400 mM, pH 9.0) to obtain liposomal solution containing 10-15% ethanol.
(3) The liposome solution obtained in step (2) was through two stacked polycarbonate membranes with pore diameter of 100 nm (100 nm+100 nm) and then two stacked polycarbonate membranes with pore diameters of 100 nm and 50 nm (100 nm+50 nm) Alternatively, the two stacked processes can be applied sequentially.
(4) The extruded liposomes were fed to a tangential flow diafiltration device and dialyzed against 8× volume of 10 mM HEPES at pH 7.6.
(5) All-trans retinoic acid (ATRA) was added to a 100 mg/mL hydroxypropyl-β-cyclodextrin (HP-β-CD) solution containing 10 mM HEPES maintained at 53° C. with magnetic stirring. The suspension was adjusted to pH to 7.6 to obtain a ATRA solution at 4 mg/mL
(6) The ATRA solution containing HP-β-CD was mixed with an equal volume of the liposome solution at the lipid concentration of 40 mg/mL. The mixture was incubated at room temperature for 30-45 minutes for drug loading, and then fed into a tangential flow diafiltration device for dialyzing against 9× volume of buffer containing 10% sucrose and 10 mM Histidine.
(7) The liposome suspension was filtered sequentially through 0.8 μm, 0.65 μm and 0.45 μm filters for storage.

### Example 2: Preparation of all-Trans Retinoic Acid Liposomes

(1) Weigh 121.742 g of hydrogenated soybean phosphatidylcholine (HSPC, molecular weight 783.8), 38.22 g of distearoylphosphatidylethanolamine-polyethylene glycol 2000 (DSPE-PEG2000), and 40.04 g of cholesterol. Dissolve them in 1.6 milliliters of ethanol and mix them in a 65° C. water bath to obtain an ethanol lipid mixture.
(2) Add the ethanol mixture obtained in step (1) into 6.4 milliliters of calcium acetate buffer solution (pH 9.0, composed of 400 mM calcium acetate and water) and incubate in a 65° C. water bath for 30 minutes to obtain liposomal vesicles.
(3) Extrude the liposomal vesicles obtained in step (2) sequentially through polycarbonate membranes with pore sizes of 200 nm, 100 nm, 80 nm, and 50 nm, each for 8 times, to finally obtain calcium acetate liposomes with an average particle size of about 75 nm in an aqueous phase.
(4) Dialyze the liposomes obtained in step (3) through a 10,000-dalton cutoff dialysis membrane in a 10% (w/w) sucrose solution with a pH of 6-7, and exchange the outer water phase of the liposomes for a 10% (w/w) sucrose solution with a pH of 6-7. The resulting calcium acetate liposomes have a phospholipid bilayer membrane and a certain pH and ion gradient between the inner and outer water phases of the bilayer. Specifically, the inner water phase of the bilayer is composed of calcium acetate solution (pH 9.0, concentration 400 mM), and the outer water phase of the bilayer is composed of sucrose solution (pH 6-7, 10% w/w).
(5) A 3.5 g all-trans retinoic acid and 0.5 mL 400 mM pH 9 carbonate buffer solution (the carbonate buffer solution was prepared. Specifically, the mass ratio of sodium carbonate and sodium bicarbonate in the buffer solution is 2.82:39.76) and 10% volume of DMSO (i.e., 10% of the volume of carbonate buffer solution, the same below) were mixed and heated at 65° C. until the drug was completely dissolved.
(6) The blank liposomes were mixed with the drug solution, and the drug was loaded by shaking at room temperature for 5 hours to obtain all-trans retinoic acid liposomes.
(7) The encapsulation rate (EE) of all-trans retinoic acid was calculated using the following formula: EE=(Wi/Wtotal)*100%, where Wi is the mass of all-trans retinoic acid in the complex, and Wtotal is the mass of all-trans retinoic acid in the same volume as before dialysis. The encapsulation rate of all-trans retinoic acid liposomes obtained was between 94% and 100. We prepared liposomes with different drug-to-lipid ratios and loading times.

TABLE 1
Formulations for Example 2.

| | | | Volume of | | | | |
|---|---|---|---|---|---|---|---|
| | | pH of | buffer | % | | Encapsulation | Drug to |
| Example | ATRA | buffer | solution | Volume | Loading | Efficiency | Lipid |
| # | (g) | solution | (mL) | of DMSO | time (h) | (%) | Ratio |
| 2 | 3.5 | 8 | 0.5 | 10 | 5 | 98 | 0.47 |
| 3 | 3 | 9 | 0.5 | 8 | 7 | 100 | 0.49 |
| 4 | 4 | 8 | 0.5 | 5 | 8 | 95 | 0.5 |
| 5 | 3 | 9 | 0.5 | 10 | 12 | 98 | 0.4 |
| 6 | 3.5 | 9 | 0.5 | 8 | 24 | 99 | 0.32 |

As shown in Table 1, the disclosed methods produced all-trans retinoic acid liposomes with relatively high drug-to-lipid ratio, and as the drug loading period increased, the encapsulated drug stability was significantly improved.

### Example 3: Preparation of all-Trans Retinoic Acid Liposomes Using Dropwise Addition Method

(1) Hydrogenated: soybean phosphatidylcholine ((HSPC, 121.742 g), distearoyl phosphatidylethanolamine-polyethylene glycol 2000 (DSPE-PEG2000, 38.22 g), and cholesterol (CHOL, 40.04 g), was dissolved in ethanol (1.6 mL) and stirred at 65° C. in a water bath to obtain an ethanol lipid mixture.
(2) The ethanol mixture obtained in step (1) was added into a calcium acetate buffer solution (6.4 mL, pH 9.0, consisting of 400 mM calcium acetate and water), and incubated in a 65° C. water bath for 30 minutes to obtain liposomal vesicles.
(3) The liposome vesicles obtained in step (2) were extruded through polycarbonate membranes with pore sizes of 200 nm, 100 nm, 80 nm, and 50 nm, respectively, for 8 times each, to obtain calcium acetate liposomes with an average particle size of about 90 nm in an aqueous phase of calcium acetate.
(4) The liposomes obtained in step (3) were dialyzed through a 10,000-dalton dialysis membrane in a 10% (w/v) sucrose solution with a pH of 6-7, replacing the external aqueous phase of the liposomes with a 10% (w/v) sucrose solution with a pH of 6-7, and obtained the calcium acetate liposomes with a phospholipid bilayer membrane and a pH gradient and ion gradient in the aqueous phase on both sides of the membrane. Specifically, the aqueous phase inside the bilayer membrane is a calcium acetate solution (pH 9.0, concentration of 400 mM), and the external aqueous phase of the bilayer membrane is a sucrose solution (pH 6-7, 10% w/v).
(5) All-trans retinoic acid (ATRA, 2 mg) was dissolved in DMSO. The drug solution was added dropwise to the prepared blank liposomes and incubated for 30 mins at 65° C. After incubation, the free all-trans retinoic acid, DMSO, and carbonate buffer was removed by dialysis through a 10000 MWCO membrane. The resulting liposome sample was filtered through a 0.22 μm filter membrane to obtain all-trans retinoic acid liposomes.
(6) The encapsulation rate (EE) of all-trans retinoic acid was calculated using the following formula: EE=(Wi/Wtotal)*100%, wherein Wi is the mass of all-trans retinoic acid in the complex, and Wtotal is the mass of all-trans retinoic acid in the same volume as before dialysis. When preparing all-trans retinoic acid liposomes with a drug-to-lipid ratio of 0.1-0.3, the obtained encapsulation efficiency was relatively low, ranging from 50% to 80%.

### Example 4: In Vitro Release Profile of all-Trans Retinoic Acid Liposomes with Different Loading Times

A 40 mg/mL bovine serum albumin (BSA) solution used to study the release profile of various formulations. The all trans-retinoic acid liposomes were mixed with a BSA solution at a volume ratio of 1:50 (60 μL ATRA-liposomes+2940 μL BSA solution) and placed in a 300 KD dialysis tube. The dialysis tube was place in 400 mL BSA medium solution. The volume ratio of full trans-retinoic acid liposomes to BSA solution in the total release system was about 1:6700. The protein binding rate of all-trans retinoic acid was 95%, the releasing free drug from the liposome immediately bound to BSA and diffused into the BSA medium out of the dialysis tube.

Residual drug was sampled and measured at 0.5 h, 2 h, 5 h, 10 h, and 24 h. By analyzing the residual ATRA content in the dialysis tube, the drug releasing character was profiled. In this way, the drug releasing profile of formulation was characterized (as drug to lipid ratio=0.35) as described in Example 1.

The results showed that all trans-retinoic acid liposomes (drug-to-lipid ratio-0.35) displayed a uniformly and stably released releasing characterize in vitro. Longer loading of the drug (e.g., 5 h and 24 h) during formation of the drug-liposome product showed greater stability.

### Example 5: Characterization of all-Trans Retinoic Acid Liposomes by Nano-DSC

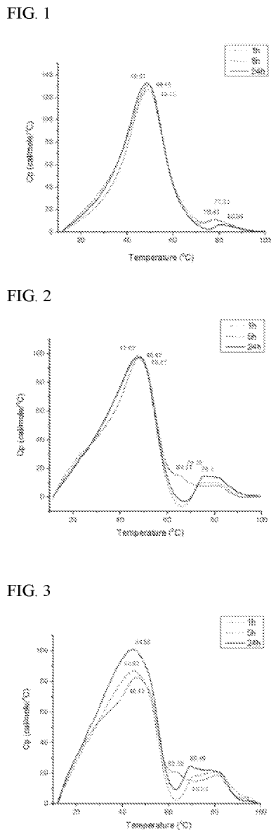

Nano-DSC was used to study the thermodynamic behavior of all-trans retinoic acid liposomes prepared via Example 1, and examined the form of the drug within the liposomes. Enthalpy is a state function. When the state of the system is fixed, then the enthalpy value is fixed. Enthalpy change is detected based on the difference between the enthalpy values of the product and reactant. A positive enthalpy change value suggests that the reaction is endothermic. The presence of 2-6% cholesterol in the C14-C20 phosphatidylcholine lipid bilayer eliminated the pre-transition and sub-transition of the phospholipid, causing the absorption peak of DSC to broaden and the enthalpy value of phase transition to decrease. Tm value is an important parameter describing the phase transition and determining the highest heat capacity change during phase transition. The lipid membrane went through a phase transition, as indicated by the Tm value associated with different melting temperature of lipid membrane from the gel state to the liquid crystal state. Generally, the peak (FIG. 1) appearing at 45° C.-55° C. shows a lipid phase transition peak. The other peak appearing at 70° C.-80° C. features the drug crystal character formed by calcium acetate and all-trans retinoic acid (in the form of microcrystals/liquid crystals/pseudo-crystals).

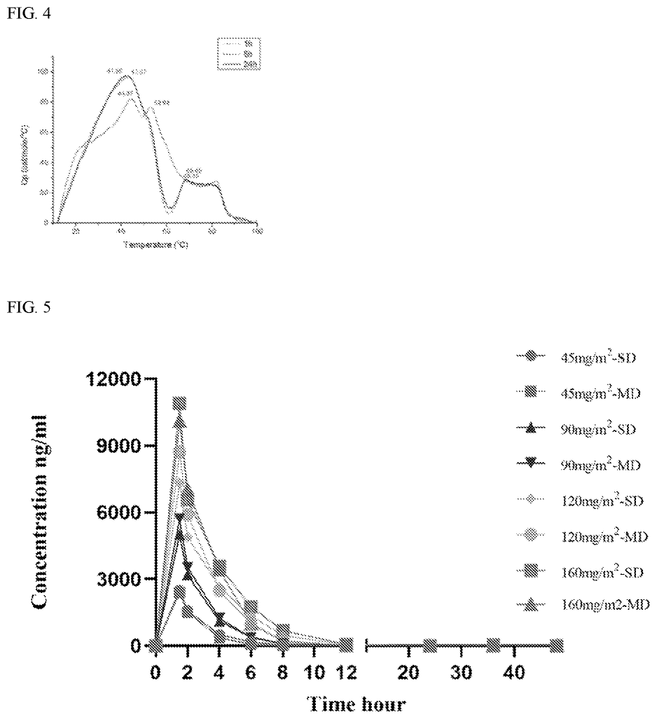

The concentration of ATRA in ATRA-liposome was fixed at 1.75 mg/mL and different drug to lipid ratios were prepared (D/L was set at 0.1, 0.2, 0.25 and 0.3) to study the crystal characteristics of ATRA. The loading periods (1 h, 5 h and 24 h) were investigated (FIGS. 1-4).

At the same ATRA concentrations, the Tm value of the drug crystal became larger as the increase of drug loading period, indicating a more stable crystal structure. As the drug-to-lipid ratio increased, phase separation occurred in the lipid peak. This might be due to the reduced stability of the liposome as the excessive drug insertion into the lipid bilayer resulted in uneven lipid rearrangement. However, with the increase of drug loading time, the phenomenon of lipid phase separation is significantly improved. Therefore, increasing the drug loading time can help to improve the stability of liposomes. Stable drug-loaded liposomes can be achieved by increasing the drug loading time when dealing with a high drug to lipid ratio formulation.

### Example 6: Safety and Pharmacokinetics

Immature myeloid cells or the so called Myeloid-derived suppressor cells (MDSCs) are found to be present in higher numbers in cancer patients' PBMCs and in tumor infiltrating cells. All-trans retinoic acid (ATRA) is a natural vitamin A metabolite that is capable of inducing MDSC maturation and differentiation.

### Method

The above described ATRA liposome formulation was used to study the tolerability and safety of ATRA in patients with refractory solid tumors based on a “3+3” dose escalation scheme. ATRA infusions are given every other day for 2 weeks followed by a break week, and the 3-week cycles were repeated until end of treatment (EOT). Safety and tolerability records, repeated dose pharmacokinetic (PK) parameters, as well as exploratory pharmacodynamics (PD) analysis of PBMC samples were evaluated during the first cycle of treatment. Efficacy evaluations were followed every 6 weeks or less until EOT.

### Results

Fifteen (15) patients were enrolled into the study in the dose groups of 45 mg/m², 90 mg/m², 120 mg/m², and 160 mg/m². The patients' primary diagnosis and prior treatment history are shown in Table 2. The ATRA liposome formulation was well tolerated at the 45 mg/m², 90 mg/m², and 120 mg/m². One patient in the 160 mg/m² group experienced a DLT (severe headache). Using the RECIST 1.1 criteria, one patient achieved complete response (CR), and four had stable disease after one cycle of treatment. The ATRA plasma concentration profiles (FIG. 5) indicated higher systemic exposure of ATRA compared to oral dosing, and there was no change of ATRA elimination after multiple doses.

TABLE 2
Patient characteristic and treatment information in this study.
Characteristic

| | | |
|---|---|---|
| Age at enrollment, year (Median, IQR) | 49.8 | (50, 11) |
| Female gender, n (%) | 3 | (20%) |
| ECOG performance status of 0 or 1, n (%) | 15 | (100%) |
| Primary Diagnosis | 15 | (100%) |
| Brain cancer | 5 | (5/15) |
| Thymic epithelial cancer | 1 | (1/15) |
| Ovarian cancer | 1 | (1/15) |
| Stomach cancer | 2 | (2/15) |
| Colorectal cancer | 2 | (2/15) |
| Lung cancer | 1 | (1/15) |
| Bile Duct Cancer | 1 | (1/15) |
| Notochordoma | 1 | (1/15) |
| Liver Cancer | 1 | (1/15) |
| Prior therapy | | |
| Surgery | 11 | (11/15) |
| Radiotherapy | 8 | (8/15) |
| Chemotherapy | 14 | (14/15) |
| Targeted therapy | 6 | (6/15) |
| Immune therapy | 5 | (5/15) |

### Example 7: Modulating MDSCs (Fixed Dose)

### Subjects

Subjects must be diagnosed with glioma by histology, and the disease has relapsed or progressed after previous treatment, and there is no effective standard treatment or the subject is intolerant to standard treatment.

Subjects must have at least one measurable lesion as defined by RECIST 1.1, e.g. at least one lesion that can be measured in two dimensions is required.

Eastern Cooperative Oncology Group (ECOG) performance status of 0 or 1. According to Karnofsky physical fitness score ≥60.

The subjects are allowed inhaled or topical corticosteroids, or hormone therapy at physiological replacement doses due to adrenal insufficiency; short-term ≤7 days) corticosteroids are allowed for prophylaxis (e.g., contrast media allergy) or treatment of non-autoimmune conditions (e.g., delayed-type hypersensitivity reactions caused by exposure to allergens); systemic corticosteroids (≥10 mg/day prednisone, or equivalent in other corticosteroids) for 7 consecutive days within 14 days of the first dose are not permitted or Immunosuppressant therapy.

### Method

Evaluate a fixed dose of ATRA in a liposome formulation on recurrent or progressive glioma, with respect to efficacy and safety in subjects with recurrent or progressive glioma. The fixed dose is given each time by intravenous infusion and the infusion time is about 60 to about 90 minutes.

Subjects were monitored throughout the study for tolerability, dose-limiting toxicities, pharmacokinetic profile and initial efficacy.

ATRA infusions were given every other day for 2 weeks, followed by a break week, totaling a 3-week cycle, and the 3-week cycle was repeated n (n>2) times until end of treatment (EOT). Safety and tolerability records, repeated dose pharmacokinetic (PK) parameters, as well as exploratory pharmacodynamics (PD) analysis of PBMC samples were evaluated during the first cycle of treatment. Efficacy evaluations were followed every 6 weeks or less until EOT.

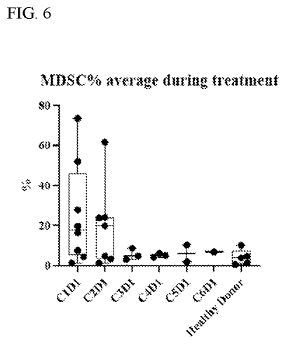

The interim circulation MDSC analysis of the glioma patients after receiving multiple treatment cycles are shown in FIG. 6. The data from a cohort of healthy donors was also included.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A method of treating cancer, comprising administering to a subject in need thereof an effective amount of all-trans retinoic acid (ATRA) formulated in a liposome, wherein the liposome comprises an inner liposome aqueous phase comprising ATRA and an aqueous solution of calcium acetate having a pH of 7 to about 10,
wherein lipid components of the liposome comprise hydrogenated soybean phosphatidylcholine (HSPC), cholesterol (CHOL), and distearoylphosphatidylethanolamine-polyethylene glycol 2000 (DSPE-PEG2000), and
wherein at least a portion of the ATRA present in the inner liposome aqueous phase is crystalline.

2. The method of claim 1, wherein the cancer is brain cancer.

3. The method of claim 2, wherein the brain cancer is a glioma.

4. The method of claim 3, wherein the glioma is oligodendroglioma or glioblastoma.

5. (canceled)

6. The method of claim 1, wherein the molar ratio of ATRA to the lipid components ranges from about 1 to about 20.

7. The method of claim 1, wherein the molar ratio of ATRA to the lipid components ranges from about 1 to about 5.

8. The method of claim 1, wherein the molar ratio between HSPC, CHOL, and DSPE-PEG2000 is in a range of (30-80):(0.1-40):(0.1-30).

9. The method of claim 1, wherein the ATRA is present in the liposome at a concentration of at least about 2 mg/mL, based on volume of the aqueous calcium acetate solution.

10. The method of claim 9, wherein the ATRA is present in the liposome at a concentration of about 2 mg/mL to about 5 mg/mL.

11. The method of claim 1, wherein the liposome has an average size of about 50 nm to about 100 nm.

12. The method of claim 11, wherein the average particle size of the liposome is about 70 nm.

13. The method of claim 1, wherein the pH of the aqueous solution of calcium acetate is about 9.

14. The method of claim 1, wherein the administering is done intravenously.

15. The method of claim 1, wherein during the course of the treating, ATRA is administered at an escalating dose of about 45 mg per m² to about 160 mg per m² of the subject's body surface area.

16. The method of claim 15, wherein ATRA is administered at an initial dose of about 45 mg per m² of the subject's body surface area.

17. The method of claim 15, wherein ATRA is administered at a subsequent dose of about 90 mg/m², about 120 mg/m², or about 160 mg/m² of the subject's body surface area.

18.-19. (canceled)

20. The method of claim 1, wherein the treating comprises at least one 3-week treatment cycle, wherein each cycle includes administering ATRA every other day for 2-weeks, followed by a 1-week off period.

21. The method of claim 20, wherein the treating comprises 2, 4, 9, or 17 treatment cycles.

22.-24. (canceled)

25. The method of claim 1, wherein the ATRA is uploaded into the liposome via a method comprising incubating a composition comprising all-trans retinoic acid (ATRA), a buffer solution, a blank liposome comprising an inner phase containing an aqueous solution of calcium acetate having a pH of about 7 to about 10, and an outer phase, external to the liposome, comprising an aqueous solution of calcium acetate having a pH of about 6 to about 7, and which is less than the pH of the inner phase of the blank liposome, and removing free all-trans retinoic acid not uploaded into the blank liposome.

26. The method of claim 25, wherein the pH of the aqueous solution of calcium acetate in the inner phase is about 10.
